(19) United States
(12) Patent Application Publication
(10) Pub. No.: US 2018/0177418 A1
(43) Pub. Date: Jun. 28, 2018

(54) PHYSIOLOGICAL INFORMATION MEASURING METHOD AND WEARABLE DEVICE

(71) Applicant: HTC Corporation, Taoyuan City (TW)

(72) Inventors: Meng-Chieh Yu, Taoyuan City (TW); Chen-Chi Wu, Taoyuan City (TW); Chung-Huan Mei, Taoyuan City (TW); Chia-Yu Yu, Taoyuan City (TW)

(73) Assignee: HTC Corporation, Taoyuan City (TW)

(21) Appl. No.: 15/389,378

(22) Filed: Dec. 22, 2016

(51) Int. Cl. A61B 5/0402; A61B 5/00; A61B 5/024; A61B 5/01; A61B 5/021; A61B 5/08; A61B 5/053

(52) U.S. Cl. CPC A61B 5/0402; A61B 5/681; A61B 5/6824; A61B 5/6826; A61B 5/02405; A61B 5/4809; A61B 5/4266; A61B 5/02108; A61B 5/0816; A61B 5/0531; A61B 5/02438; A61B 5/01

(57) ABSTRACT

A physiological information measuring method and a wearable device are provided. The physiological information measuring method includes measuring a physiological information of a user in a discontinuous manner by a wearable device worn on the user when the user is in a sleep state. The wearable device is configured to perform the physiological information measuring method.

### BACKGROUND OF THE INVENTION

### 1. Field of the Invention

The application generally relates to a measuring method and a device, and particularly, to a physiological information measuring method and a wearable device.

### 2. Description of Related Art

With advances in technology, various types of physiological information such as respiration rate, heart rate, blood pressure value and so forth are no longer limited to be measured by professional instruments, and locations where measurements may be taken also do not have to cope with large-scale instruments by only being set at medical institutions or other indoor environments. Currently, there are already many wearable devices targeting health management on the market. This type of device is not only light and easy to wear but also include a variety of physiological information measuring functions, and may closely measure and record various types of physiological information of a user at anytime and anywhere. Afterwards, the information being collected may also be analysed and collated directly on the wearable device or be transmitted to a server through the network to perform integration and analysis, thereby enabling the user to know his/her own health status.

Some of the physiological information may only be obtained when the user is in a sleep state or after a long-time rest. In other to obtain those types of physiological information, an approach of conventional wearable devices is to perform the measurements during the sleep state; however, such measuring approach leads to excessive power consumption of the wearable devices. Another approach of the conventional wearable devices is to require the user to sit still for 10 minutes before performing the measurements; however, such measuring approach is not practical for modern people who demand efficiency in everything.

### SUMMARY OF THE INVENTION

The application provides a physiological information measuring method and a wearable device capable of resolving the problems with conventional measuring methods being power-consuming and non-practical.

The physiological information measuring method of the application includes measuring a physiological information of a user by a wearable device worn by the user in a discontinuous manner when the user is in a sleep state.

In one embodiment of the application, the step of measuring the physiological information in the discontinuous manner includes fixed periodically measuring the physiological information.

In one embodiment of the application, the step of measuring the physiological information in the discontinuous manner includes: according to an alarm time set by the user on the wearable device, measuring the physiological information before the alarm time.

In one embodiment of the application, the step of measuring the physiological information in the discontinuous manner includes: according to an average sleep cycle of the user stored in the wearable device, measuring the physiological information before the ending of the average sleep cycle.

In one embodiment of the application, the step of measuring the physiological information in the discontinuous manner includes: according to an average wake up time of the user stored in the wearable device, measuring the physiological information before the advent of the average wake up time.

In one embodiment of the application, the physiological information is one of a resting heart rate, a body temperature, a perspiration rate, an electrocardiography, a photoplethysmography, a heart rate variability, a blood pressure value, a pulse wave velocity, a respiratory rate, and a skin conductance.

The wearable device of the application includes a processing unit and a measuring unit. The measuring unit is coupled to the processing unit. A physiological information of a user is measured by the measuring unit in a discontinuous manner when the user is in a sleep state.

In one embodiment of the application, the processing unit includes a storage unit configured to store a measurement parameter. The processing unit drives the measuring unit to measure the physiological information of the user according to the measurement parameter.

In one embodiment of the application, the measuring unit is an electrocardiogram measuring unit.

In one embodiment of the application, the wearable device further includes a wireless transmission module coupled to the processing unit and configured to wireless transmit the physiological information or a measurement parameter.

In one embodiment of the application, the wearable device is a watch, a wristband or a ring.

In view of the above, in the physiological information measuring method and the wearable device of the application, power consumption may be greatly reduced and the convenience of use may be greatly enhanced.

In order to make the aforementioned features and advantages of the application more comprehensible, embodiments accompanying figures are described in detail below.

### BRIEF DESCRIPTION OF THE DRAWINGS

The accompanying drawings are included to provide a further understanding of the application, and are incorporated in and constitute a part of this specification. The drawings illustrate embodiments of the application and, together with the description, serve to explain the principles of the application.

FIG. 1 is a time flowchart illustrating a physiological information measuring method according to an embodiment of the application.

FIG. 2A is a schematic diagram illustrating a wearable electronic device configured to execute the physiological information measuring method of the application.

FIG. 2B is a schematic diagram illustrating a circuit architecture of the wearable electronic device according to an embodiment of the application.

FIG. 3 is a time flowchart illustrating a physiological information measuring method according to another embodiment of the application.

FIG. 4 is a time flowchart illustrating a physiological information measuring method according to yet another embodiment of the application.

FIG. 5 is a time flowchart illustrating a physiological information measuring method according to still another embodiment of the application.

### DESCRIPTION OF THE EMBODIMENTS

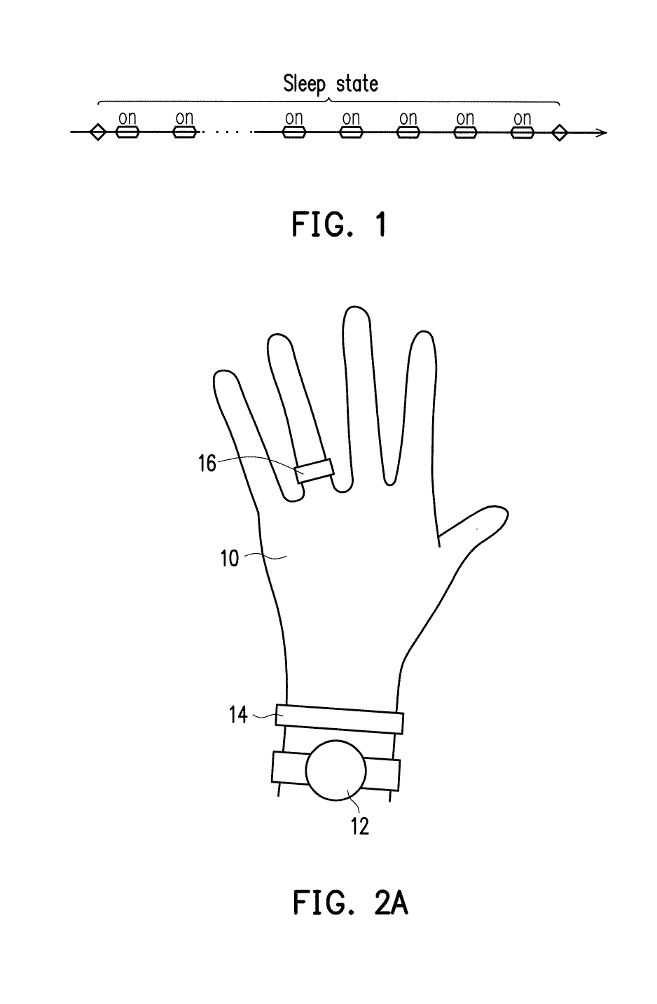

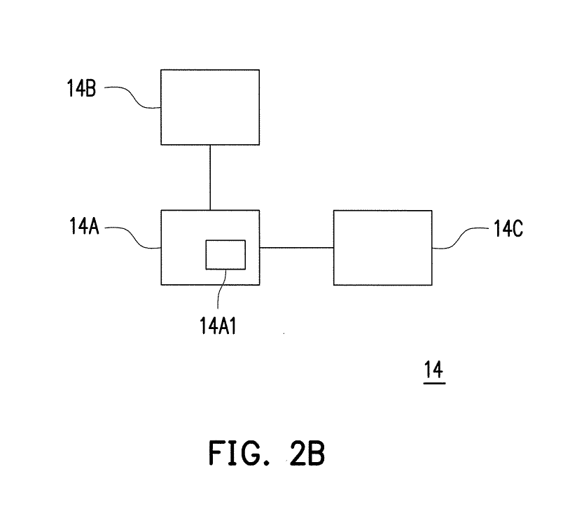

FIG. 1 is a time flowchart illustrating a physiological information measuring method according to an embodiment of the application. FIG. 2A is a schematic diagram illustrating a wearable electronic device configured to execute the physiological information measuring method of the application. Referring to FIG. 1 and FIG. 2A, the physiological information measuring method of the present embodiment is to measure a physiological information of a user 10 by a wearable device 12 worn on the user 10 in a discontinuous manner when the user 10 is in a sleep state. In other words, during the sleep state of the user 10, the wearable device 14, instead of being entirely maintained in the state of measuring the physiological information, measures the physiological information in the discontinuous manner. Thus, the wearable device 14 does not need to consume a large amount of electric energy and may prolong a usage time of the wearable device 14 after each charge, thereby providing the user 10 with convenient use experience. Moreover, since the measurement is completed during the sleep state, the user 10 is not required to sit still for a long period of time in order for the measurement to be performed, and thus there is no time burden on the user 10.

For example, the physiological information may be a resting heart rate. The resting heart rate refers to the number of heartbeats per minute during inactive resting state, and thus the sleep state is a better time for performing the measurement. In general, it is more ideal for the heart rate of a human body under a resting state to be 55˜70 bpm (beats/minute), and more attention should be paid if it often exceeding 80˜85 bpm. Moreover, the physiological information may also be a body temperature, a perspiration rate, an electrocardiography (ECG), a photoplethysmography (PPG), a heart rate variability, a blood pressure value, a pulse wave velocity, a respiratory rate, a skin conductance (SC), or other physiological information.

In the foregoing description, the wearable device 14 of the present embodiment is, for example, a wristband 14, but a watch 12 and a ring 16 in FIG. 2A may also be used to perform the physiological information measuring method. In addition, a chest wearing device, a head mounted device or other wearable devices may also be used to perform the physiological information measuring method, and thus may also be the wearable device of the application. Any of the following or other physiological information measuring methods of the application may all be performed with the wearable device of the application as discussed herein.

In the present embodiment, the measuring of the physiological information in the discontinuous manner is to fixed periodically measure the physiological information. For example, the measuring function of the wearable device 14 may be placed in an ON-state to perform 1 minute of measurement for every 10 minutes. Afterwards, when the wearable device 14 detects the user 10 is awakened, then a last measurement result may be stored as the resting heart rate. Certainly, the so-called discontinuous manner may also be a non-fixed periodical measuring approach, such as gradually reducing the measurement interval or other means.

In the following, a circuit architecture configuration of the wearable device 14 of the present embodiment is described with reference to FIG. 2B; however, those of ordinary skill in the art with reference to the foregoing descriptions should be able to understand that measurements may be performed using a variety of possible circuit implementations depending on the measurement requirements of different types of physiological information. Certainly, the circuit architecture of the wearable device 14 of FIG. 2B may also be applied to the watch 12 or the ring 16 of FIG. 2A, or the wearable devices of other embodiments of the application. Referring to FIG. 2B, the wearable device 14 includes a processing unit 14A and a measuring unit 14B. The measuring unit 14B is coupled to the processing unit 14A. The processing unit 14A may include a storage unit 14A1 configured to store a measurement parameter. The measurement parameter is, for example, includes a time information, and the processing unit 14A drives the measuring unit 14B to detect the physiological information of the user according to the time information included by the measurement parameter. The measuring unit 14B may be used to detect a single physiological information or different kinds of physiological information. The measuring unit 14B may be corresponded to a detector for detecting the physiological information, such that the measuring unit 14B may be an ECG measuring unit configured to measure the heart conditions. The physiological information may be a resting heart rate, a body temperature, a perspiration rate, an electrocardiography (ECG), a photoplethysmography (PPG), a heart rate variability, a blood pressure value, a pulse wave velocity, a respiratory rate, a skin conductance (SC), or other physiological information.

The wearable device 14 may further include a wireless transmission module 14C configured to wirelessly transmit the physiological information. The physiological information measured by the wearable device 14 may be transmitted to a monitoring terminal device (not shown) by means of wired or wireless transmission, so that the user may check the measured physiological information from the monitoring terminal device. When the wireless transmission module 14C is not connected with the terminal device, the physiological information may also be stored in the storage unit 14A1. After the wireless transmission module 14C is connected with the terminal device, the physiological information stored in the storage unit 14A1 may be transmitted to the terminal device. The wearable device 14 and the monitoring terminal device may transmit signals to each other through a wired transmission interface or a wireless transmission interface. The wearable device 14 of the present embodiment may be configured with corresponding circuit elements depending on the requirements of function. For example, the wearable device 14 may further include a timing circuit and a display module so as to provide a time information display function, but the application is not limited thereto. The monitoring terminal device is, for example, any type of an electronic device such as a server, a desktop computer, a notebook computer, a tablet PC, a smartphone or so forth. Certainly, the wearable device 14 itself may also have information analysis and collation functions, and may further directly display the analysed and collated information via the display module for the user to understand.

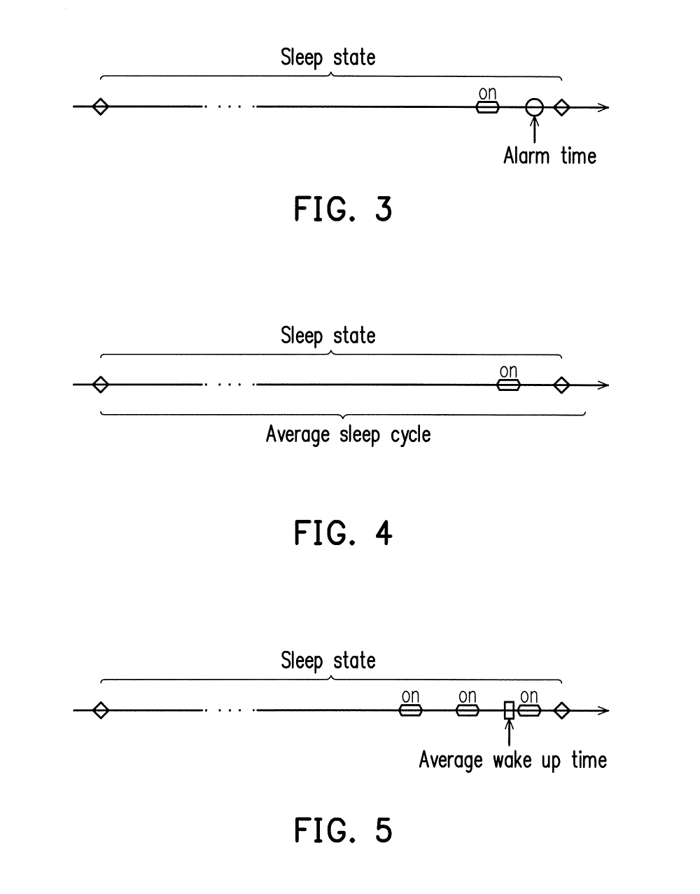

FIG. 3 is a time flowchart illustrating a physiological information measuring method according to another embodiment of the application. Referring to FIG. 2A, FIG. 2B and FIG. 3, the physiological information measuring method of the present embodiment is similar to that of the embodiment in FIG. 1, and a difference lies in that the measurement of the physiological information in the discontinuous manner of the present embodiment includes: according to an alarm time set by the user 10 on the wearable device 14, measuring the physiological information before the alarm time. Generally, when the wearable device 14 provides an alarm clock function, the user 10 may set an alarm time to avoid being late for work or other appointments, and thus the sleep state of the user 10 may most likely be ended at the alarm time or later. Therefore, it may be set to perform the measurement continuously, fixed periodically, non-fixed periodically, or with other rules starting from a half hour, one hour or other time before the alarm time till the wearable device 14 detects that the user 10 is awakened. The alarm time may be set by the user directly on the wearable device 14 and be stored in the storage unit 14A1. The alarm time is included within the measurement parameter, and the measurement parameter may also be obtained by an external device, such as a mobile phone, a server; otherwise, hospital physicians may also use a computer device to wiredly or wirelessly connect to the wearable device 14 to set the measurement parameter. The server may be stored with schedule of the user or periodic time settings. When the wearable device 14 obtains the measurement parameter through the wireless transmission module 14C and stores it in the storage unit 14A1, the processing unit 14A sets a measuring time according to the measurement parameter. When the measuring time comes, the processing unit 14A drives the measuring unit 14B to measure the physiological information.

FIG. 4 is a time flowchart illustrating a physiological information measuring method according to yet another embodiment of the application. Referring to FIG. 2A, FIG. 2B and FIG. 4, the physiological information measuring method of the present embodiment is similar to that of the embodiment of FIG. 1, and a difference lies in that the measurement of the physiological information in the discontinuous manner of the present embodiment includes: the processing unit 14A driving the measuring unit 14B to continuously or periodically measure the physiological information throughout the day. The processing unit 14A stores the measured physiological information in the storage unit 14A 1. The aforementioned step of continuously or periodically measuring the physiological information throughout the day may be to perform the measurement every 5 minutes throughout the day, and to store 288 times (24 hours×each hour having 12 times of 5 minutes) of measurement data in the storage unit 14A 1 of the processing unit 14A. According to the periodically measured physiological information of the user 10 as stored by the wearable device 14, a measurement parameter is determined. The measurement parameter may include a physiological cycle of the user, such as an average sleep cycle. The processing unit 14A generates a measuring time according to the measurement parameter, and the measuring time is a time of measuring the physiological information of the user before the ending of a physiological cycle (average sleep cycle). Generally, when the wearable device 14 provides the sleep detection function, the wearable device 14 may obtain the average sleep cycle of the user 10 and estimates the ending time of the sleep state of the user 10. Therefore, it may be set to perform the measurement continuously, fixed periodically, non-fixed periodically, or with other rules starting from a half hour, one hour or other time before the ending time till the wearable device 14 detects that the user 10 is awakened. For instance, the measurement parameter of the user is the physiological cycle (average sleep cycle) of the user, and the physiological cycle (average sleep cycle) is from 10 O'clock at the night to 8 O'clock in the next day. The processing unit 14A generates the measuring time according to the measurement parameter (8 O'clock in the morning). The measuring time may be set as to start measuring the physiological information at 10 minutes earlier (7:50).

FIG. 5 is a time flowchart illustrating a physiological information measuring method according to still another embodiment of the application. Referring to FIG. 2A, FIG. 2B and FIG. 5, the physiological information measuring method of the present embodiment is similar to that of the embodiment of FIG. 1, and a difference lies in that the measurement of the physiological information in the discontinuous manner of the present embodiment includes: the processing unit 14A driving the measuring unit 14B to continuously or periodically measure the physiological information throughout the day. The processing unit 14A stores the measured physiological information in the storage unit 14A1. According to the periodically measured physiological information of the user 10 as stored by the wearable device 14, a measurement parameter is determined. The measurement parameter may include a physiological cycle of the user, such as an average wake up time, and the physiological information is measured before the advent of the average wake up time. Generally, when the wearable device 14 provides the sleep detection function, the wearable device 14 may obtain the average wake up time of the user 10, namely, may estimate the ending time of the sleep state of the user 10. The processing unit 14A generates the measuring time according to the measurement parameter, and the measuring time is a time for measuring the physiological information of the user before the advent of the physiological cycle (average wake up time). Therefore, it may be set to perform the measurement continuously, fixed periodically, non-fixed periodically, or with other rules starting from a half hour, one hour or other time before the average wake up time till the wearable device 14 detects that the user 10 is awakened. For instance, the measurement parameter of the user is the physiological cycle (average wake up time) of the user, and the physiological cycle (average wake up time) is at 8 O'clock in the morning. The processing unit 14A generates the measuring time according to the measurement parameter (8 O'clock in the morning). The measuring time may be set as to start measuring the physiological information at 10 minutes earlier (7:50).

In summary, in the physiological information measuring method and the wearable device of the application, the wearable device worn on the user measures the physiological information of the user in the discontinuous manner during the sleep state, and thus may greatly reduce the power consumption and may also greatly enhance the convenience of use by directly measuring the desired information during the sleep state.

It will be apparent to those skilled in the art that various modifications and variations may be made to the structure of the present application without departing from the scope or spirit of the application. In view of the foregoing, it is intended that the present application cover modifications and variations of this application provided they fall within the scope of the following claims and their equivalents.

What is claimed is:

1. A physiological information measuring method, comprising:
measuring a physiological information of a user in a discontinuous manner by a wearable device worn on the user when the user is in a sleep state.

2. The physiological information measuring method as recited in claim 1, wherein the step of measuring the physiological information in the discontinuous manner comprises fixed periodically measuring the physiological information.

3. The physiological information measuring method as recited in claim 1, wherein the step of measuring the physiological information in the discontinuous manner comprises:
according to an alarm time set by the user on the wearable device, measuring the physiological information before the alarm time.

4. The physiological information measuring method as recited in claim 1, wherein the step of measuring the physiological information in the discontinuous manner comprises:
according to an average sleep cycle of the user stored in the wearable device, measuring the physiological information before the ending of the average sleep cycle.

5. The physiological information measuring method as recited in claim 1, wherein the step of measuring the physiological information in the discontinuous manner comprises:
according to an average wake up time of the user stored in the wearable device, measuring the physiological information before the advent of the average wake up time.

6. The physiological information measuring method as recited in claim 1 wherein the physiological information is one of a resting heart rate, a body temperature, a perspiration rate, an electrocardiography, a photoplethysmography, a heart rate variability, a blood pressure value, a pulse wave velocity, a respiratory rate, and a skin conductance.

7. A wearable device, comprising:
a processing unit; and
a measuring unit, coupled to the processing unit, wherein a physiological information of a user is measured by the measuring unit in a discontinuous manner when the user is in a sleep state.

8. The wearable device as recited in claim 7, wherein the processing unit comprises a storage unit configured to store a measurement parameter, and the processing unit drives the measuring unit to measure the physiological information of the user according to the measurement parameter.

9. The wearable device as recited in claim 7, wherein the measuring unit is an electrocardiogram measuring unit.

10. The wearable device as recited in claim 7, further comprising a wireless transmission module coupled to the processing unit and configured to wireless transmit the physiological information or a measurement parameter.

11. The wearable device as recited in claim 7, being a watch, a wristband or a ring.
